(19) United States
(12) Patent Application Publication
(10) Pub. No.: US 2013/0165758 A1
(43) Pub. Date: Jun. 27, 2013

(54) MINIATURE INFUSION PUMP

(71) Applicants: Dale A. Young, Lake Elmo, MN (US); John M. Gray, Brooklyn Park, MN (US); Alex O. Espe, Minneapolis, MN (US)

(72) Inventors: Dale A. Young, Lake Elmo, MN (US); John M. Gray, Brooklyn Park, MN (US); Alex O. Espe, Minneapolis, MN (US)

(73) Assignee: Medtronic, Inc., Minneapolis, MN (US)

(21) Appl. No.: 13/333,441

(22) Filed: Dec. 21, 2011

(51) Int. Cl. A61B 5/145

(52) U.S. Cl. 600/366

(57) ABSTRACT

A miniature infusion pump (MIP) suitable for use with a human patient is configured for non-human animal testing. In one example, a MIP includes an electromagnetic piston pump, a circuit board, and a housing. The pump is configured to deliver a therapeutic agent from a reservoir through an implantable catheter. The circuit board includes programmable electronics configured to control the pump to deliver the therapeutic agent through the catheter. The pump and the circuit board are sealed within the housing. The MIP is sized to be at least one of harnessed to or implanted in a non-human test subject including a weight greater than or equal to approximately 150 to approximately 250 grams.

### TECHNICAL FIELD

This disclosure relates to implantable medical devices and, more particularly, to implantable infusion devices that may be employed in pre-clinical studies with small non-human animals.

### BACKGROUND

A variety of medical devices are used for chronic, i.e., long-term, delivery of fluid therapy to patients suffering from a variety of conditions, such as chronic pain, tremor, Parkinson's disease, epilepsy, urinary or fecal incontinence, sexual dysfunction, obesity, spasticity, or gastroparesis. For example, pumps or other fluid delivery devices can be used for chronic delivery of therapeutic agents, such as drugs to patients. These devices are intended to provide a patient with a therapeutic output to alleviate or assist with a variety of conditions. Typically, such devices are implanted in a patient and provide a therapeutic output under specified conditions on a recurring basis.

One type of implantable fluid delivery device is a drug infusion device that can deliver a drug or other therapeutic agent to a patient at a selected site. A drug infusion device may be partially or completely implanted at a location in the body of a patient and deliver a fluid medication through a catheter to a selected delivery site in the body. Drug infusion devices, such as implantable drug pumps, commonly include a reservoir for holding a supply of the therapeutic agent, such as a drug, for delivery to a site in the patient. The fluid reservoir can be self-sealing and accessible through one or more ports. A pump is fluidly coupled to the reservoir for delivering the therapeutic agent to the patient. A catheter provides a pathway for delivering the therapeutic agent from the pump to the delivery site in the patient.

### SUMMARY

The present disclosure is directed to miniature infusion pumps suitable for use with a human patient that are configured for non-human animal testing. In one example, a miniature infusion pump according to this disclosure includes a cylindrical housing, an electromagnetic piston pump, a circuit board, and a conduit, and a reservoir junction. The electromagnetic piston pump is configured to deliver a therapeutic agent from a reservoir through an implantable catheter. The circuit board includes programmable electronict configured to control the pump to deliver the therapeutic agent through the catheter. The pump and the circuit board are arranged in stacked relationship to one another within the cylindrical housing such that the pump is arranged toward one end of the cylindrical housing and the circuit board is arranged toward an opposite end of the cylindrical housing. The conduit is interposed between the pump and the circuit board and is configured to fluidically connect an outlet of the pump to the implantable catheter. The reservoir junction is connected to the end of the housing toward which the pump is arranged. The reservoir junction is configured to fluidically connect an inlet of the pump to a reservoir configured to store the therapeutic agent.

In another example, a miniature infusion pump according to this disclosure includes an electromagnetic piston pump, a circuit board, and a housing. The electromagnetic piston pump is configured to deliver a therapeutic agent from a reservoir through an implantable catheter. The circuit board includes programmable electronics configured to control the pump to deliver the therapeutic agent through the catheter. The pump and the circuit board are sealed within the housing. The miniature infusion pump is sized to be at least one of harnessed to or implanted in a non-human test subject comprising a weight greater than or equal to approximately 150 to approximately 250 grams.

Another example includes a method of using a miniature infusion pump suitable for use with a human patient for non-human animal testing. The method includes implanting at least a portion of a catheter within a non-human test subject and delivering a dose of the therapeutic agent to the test subject through the catheter with the miniature infusion pump. The catheter is coupled to the miniature infusion pump. The miniature infusion pump includes an electromagnetic piston pump, a circuit board, and a housing. The electromagnetic piston pump is configured to deliver a therapeutic agent from a reservoir through an implantable catheter. The circuit board includes programmable electronics configured to control the pump to deliver the therapeutic agent through the catheter. The pump and the circuit board are sealed within the housing. The miniature infusion pump is sized to be at least one of harnessed to or implanted in a non-human test subject comprising a weight greater than or equal to approximately 150 to approximately 250 grams.

The details of one or more examples are set forth in the accompanying drawings and the description below. Other features, objects, and advantages of examples according to this disclosure will be apparent from the description and drawings, and from the claims.

### BRIEF DESCRIPTION OF DRAWINGS

FIG. 1 is a conceptual diagram illustrating an example of a fluid delivery system including a miniature infusion pump configured to deliver a therapeutic agent to a test subject via a catheter.

FIG. 2 is functional block diagram illustrating an example of the miniature infusion pump of FIG. 1.

FIG. 3 is a perspective view of an example miniature infusion pump according to this disclosure.

FIG. 4 is a partially exploded view illustrating the example miniature infusion pump of FIG. 3.

FIG. 5 is a section view illustrating the example miniature infusion pump of FIG. 3.

FIGS. 6A and 6B are plan and elevation views of another example miniature infusion pump according to this disclosure.

FIG. 7 is a flow chart illustrating an example method of using a miniature infusion pump suitable for use with a human patient for non-human animal testing.

### DETAILED DESCRIPTION

Prior to delivering a new therapeutic agent to a human via an implantable infusion device (IID), e.g. a drug infusion pump, e.g. during clinical trials conducted as part of regulatory (e.g. an Federal Drug Administration) procedure, the new therapeutic agent is subjected to a great deal of testing, including animal testing. Therapeutic agents that are intended to be delivered to human subjects via an IID are commonly tested using specialized miniature infusion pumps (MIPs) designed for such pre-clinical animal testing procedures. A number of different designs exist, including peristaltic and osmotic pumps, but a common characteristic of such MIPs is that the devices generally include a less robust design than IIDs that are suitable for use with a human patient, e.g., suitable for human implantation in order to reduce costs during these preliminary testing stages.

For example, some MIPs employed in pre-clinical animal testing may include less expensive and lower quality materials than those materials employed in IIDs. These MIPs may also be designed for single use, meaning that the fluid delivery capacity, both in terms of pump cycle frequency and cumulative fluid delivery capacity may be intentionally limited to capacities needed for a single, relatively short term animal trial. As these devices may only be designed for single use, they may also be made from materials and designed such that they may not be resterilized after uses. Additionally, some MIPs employed in pre-clinical animal testing have commonly included limited, if any, programmability. Some such devices may, e.g., either be mechanically configured to deliver a certain amount of therapeutic agent to a test subject without any digital control, or may be programmed once with a single delivery regime that will dictate delivery without changes throughout a trial.

At first glance, it may seem intuitive that reducing costs during preliminary pre-clinical tests is generally desirable. However, there may be untoward consequences of the MIPs employed in such procedures having less robust and different designs than the IIDs in which the therapeutic agent may eventually be employed in conjunction with human implantation. One consequence of the lack of identity of structure between the animal test MIPs and IIDs configured for use with a human patient may be that therapeutic agents that graduate from pre-clinical animal tests may need to be retested for compatibility with an IID device independently after such tests. One reason that may necessitate retesting of a therapeutic agent is that the materials of the IID with which the therapeutic agent interacts are different than the MIP and therefore the agent needs to be validated with these materials to ensure there are not any undesirable or unsafe interactions there between.

For example, MIPs employed in animal testing are commonly fabricated from non-metallic materials including various polymers, while IID pumps may be fabricated from biocompatible metals including titanium. Because certain therapeutic agents may interact differently with different materials, the agent may need to be tested in the IID in spite of the earlier testing in the MIP. In other words, while the pre-clinical animal testing may deliver the agent to test subjects in a similar fashion as will be employed in human implantation, a later test may be necessary to test the compatibility of the agent with the IID independent of the therapeutic efficacy of the agent in treating the patient discovered during the animal testing.

Thus, while reduced cost and less robust designs may at first seem advantageous for pre-clinical animal testing MIPs, employing similar designs as those used for human patients applications may save time and money over the course of an entire approval process for a new therapeutic agent. For example, employing similar devices during animal and human testing may reducing or eliminate the need for the redundant step of testing therapeutic agent and device compatibility with human patients after pre-clinical animal testing of the efficacy of the therapeutic agent. In view of the foregoing challenges of testing new therapeutic agents for use with human patients, examples according to this disclosure include a MIP that is suitable for use with a human patient configured for non-human animal testing.

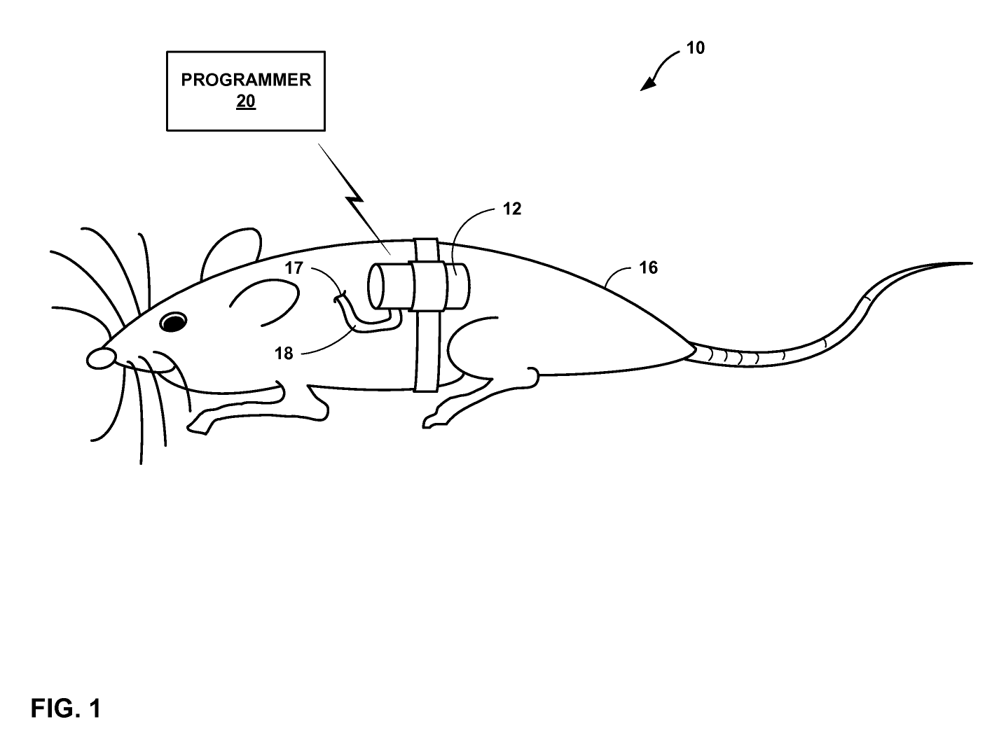

FIG. 1 is a conceptual diagram illustrating an example of a therapy system 10, which includes MIP 12, catheter 18, and external programmer 20. MIP 12 is connected to catheter 18 to deliver at least one therapeutic agent, e.g. a pharmaceutical agent, pain relieving agent, anti-inflammatory agent, gene therapy agent, or the like, to a target site within test subject 16. MIP 12 is designed to be employed for pre-clinical testing with small non-human animals. In FIG. 1, test subject 16 includes a medium-sized rodent, which may include various strains of rats (rattus norvegicus), including, e.g. Wister, Sprague Dawley, Long-Evans, and Zucker rats. In other examples, MIPs according to this disclosure may be employed with other small non-human mammals, including, e.g. rabbits (oryctolagus cuniculus), like New Zealand and Dutch breed rabbits.

MIP 12 includes an outer housing that is constructed of a biocompatible material that resists corrosion and degradation from bodily fluids including, e.g., titanium or biologically inert polymers. In the example of FIG. 1, MIP 12 is harnessed to test subject 16 and catheter 18 is a percutaneous catheter connected between MIP 12 and a target delivery site within test subject 16. In other examples, MIP 12 may be implanted within a subcutaneous pocket within test subject 16, e.g. relatively close to the therapy delivery site. In other examples, MIP 12 may be implanted within other suitable sites within test subject 16, which may depend, for example, on the target site within test subject 16 for the delivery of the therapeutic agent.

MIP 12 delivers a therapeutic agent from a reservoir (not shown) to test subject 16 through catheter 18 from a proximal end coupled to MIP 12 to a distal end located proximate to the target site within test subject 16. Example therapeutic agents that may be delivered by MIP 12 to test subject 16 during pre-clinical non-human trials include, e.g., insulin, morphine, hydromorphone, bupivacaine, clonidine, other analgesics, baclofen and other muscle relaxers and antispastic agents, genetic agents, antibiotics, nutritional fluids, hormones or hormonal drugs, gene therapy drugs, anticoagulants, cardiovascular medications or chemotherapeutics.

Catheter 18 can comprise a unitary catheter or a plurality of catheter segments connected together to form an overall catheter length. External programmer 20 is configured to wirelessly communicate with MIP 12 as needed, such as to provide or retrieve therapy information or control aspects of therapy delivery (e.g., modify the therapy parameters such as rate or timing of delivery, turn MIP 12 on or off, and so forth) from MIP 12 to test subject 16.

Catheter 18 may be coupled to MIP 12 either directly or with the aid of a catheter extension (not shown in FIG. 1). In the example shown in FIG. 1, catheter 18 traverses from the location at which MIP 12 is harnessed to test subject 16 through incision 17 to one or more targets sites within the test subject. Catheter 18 is positioned such that one or more fluid delivery outlets (not shown in FIG. 1) of catheter 18 are proximate to the targets within test subject 16, e.g. in or near the brain, spinal cord, and various peripheral nerves like vagus and sacral nerves of the test subject. In some examples, multiple catheters may be coupled to MIP 12 to target the same or different nerve or other tissue sites within test subject 16, or catheter 18 may include multiple lumens to deliver multiple therapeutic agents to the test subject.

Programmer 20 is an external computing device that is configured to communicate with MIP 12 by wireless telemetry. For example, programmer 20 may be a programmer that a clinician conducting the tests with test subject 16 uses to communicate with MIP 12 and program therapy delivered by the MIP. Programmer 20 may be a handheld or other dedicated computing device, or a larger workstation or a separate application within another multi-function device.

MIP 12 is a device that is suitable for use with a human patient, e.g., suitable for human implantation but is configured for non-human animal testing. MIP 12 includes an electromagnetic piston pump, a circuit board, and a housing within which the pump and the circuit board are sealed. The electromagnetic piston pump is configured to deliver a therapeutic agent from a reservoir through an implantable catheter. The circuit board includes programmable electronics configured to control the pump to deliver the therapeutic agent through the catheter. The MIP is sized to be at least one of harnessed to or implanted in test subject 16 without substantially altering behavior of the subject. Although test subject 16 includes a rat, in examples according to this disclosure the non-human test subject for which MIP 12 or other MIPs according to this disclosure are sized includes a weight greater than or equal to approximately 150 to approximately 250 grams, which may include mammals ranging from medium-sized rodents up to primates. One of the challenges of pre-clinical testing of non-human test subjects is employing devices that will not substantially alter the behavior of the subjects. For example, some pumps that deliver agents to test subjects are tethered to the subject by a long catheter. In such circumstances, there is a risk that the presence of such a device may alter the normal behavior of the subject, which, in turn, may affect the results of the test. As such, MIPs according to this disclosure may be sized relative to the size of the test subjects with which they are employed such that the subjects are not aware of or quickly become accustomed to the presence of the device, whether harnessed to or implanted within the subjects. In one example, MIP 12 includes a weight of approximately 30 grams and a volume of approximately 12.6 cubic centimeters (0.77 cubic inches).

Although employed for non-human mammal testing with subject 16, MIP 12 is suitable for use with a human patient, which may mean that the materials, fluid delivery capacity, and/or longevity of MIP 12 are suitable for use in conjunction with an IID implanted within a human patient. For example, the pump and other components of MIP 12 that interact with the therapeutic agent may be fabricated from materials suitable for use in humans, including titanium. Additionally, the pump may be capable of delivering high frequency pump strokes over long periods of time. For example, prior MIPs employed in pre-clinical animal studies have included a 1 milliliter reservoir and were configured to deliver enough fluid to a test subject to refill the reservoir 1-3 times, totaling, at most, approximately 3 milliliters of fluid over the life of the device in a single animal test. MIP 12, however, may be designed to deliver high frequency nominal 1 microliter pump strokes at varying programmable rates over periods of time ranging from a few months (range of time appropriate for animal testing) to five or more years (range of time appropriate for human implantation). The actual pump stroke volume of MIP 12 may be in a range, e.g., from approximately 0.6 microliters to approximately 1.1 microliters. The electromagnetic pump of MIP 12 may be configured to deliver as many as 2.5 million pump stroke cycles, totaling approximately 2.5 liters of therapeutic agent, or 2500 milliliters of the agent. A single animal test employing a MIP may include delivery of on the order of approximately 2-3 milliliters of therapeutic agent over the course of the study, in some examples like rat studies. Thus, while previous MIPs employed in pre-clinical animal testing have generally had a longevity and capacity roughly equal to a single animal test, MIP 12 may be configured to deliver a cumulative 2500 milliliters of agent which may be used in more than 850 animal tests. Because MIP 12 is configured to be resterilized, the device may be harnessed to or implanted in a number of test subjects and resterilized in between in order to be employed across a large number of animal tests. Thus, although the initial cost of MIP 12 may be greater than prior less robust MIPs employed in pre-clinical non-human testing, the longevity of MIP 12 may allow for partial or complete recovery of the up-front costs of the device.

In addition to the cumulative delivery capacity of MIP 12, the device may also exhibit an increased daily capacity compared to past devices. For example, MIP 12 may be configured to deliver up to approximately 12 milliliters of therapeutic agent per day, while prior pumps are commonly limited to daily capacities on the order of approximately 1 milliliter. The increased daily capacity of MIP 12 may be beneficial because in order to deliver the correct dose to a test subject with prior pumps within a certain period of time, e.g. a day, a very high concentration of the agent is needed. However, high concentrations of some therapeutic agents can cause problems with the agent precipitating out of the solution. Thus, by running at a higher flow rate and increased daily capacity, MIP 12 may allow the concentration of the therapeutic agent to be less than previously possible for the same dose, thereby avoiding or reducing the risk of precipitation problems.

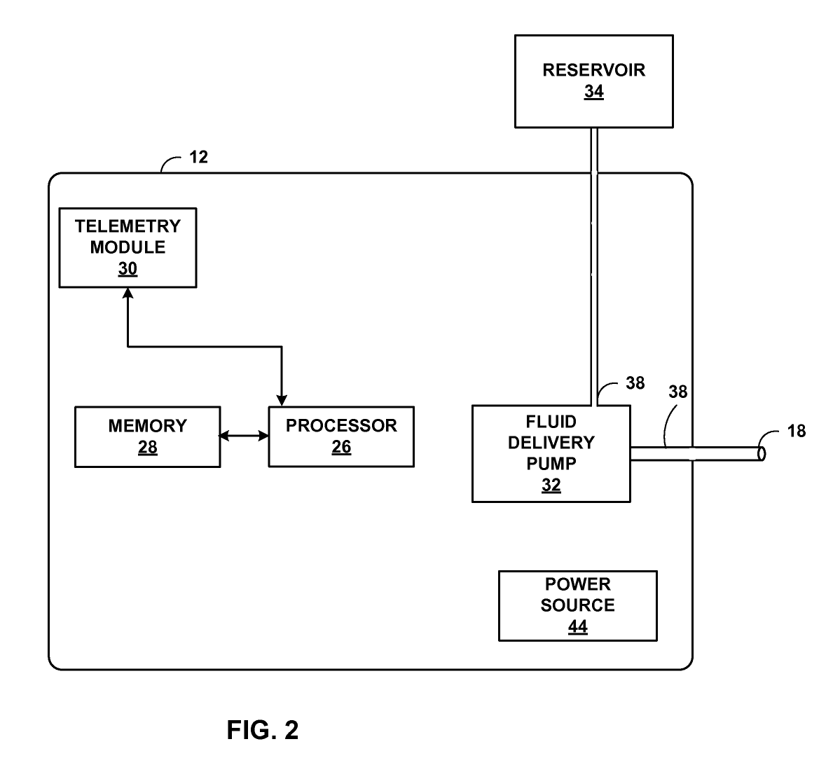

FIG. 2 is a functional block diagram illustrating components of an example of MIP 12, which includes processor 26, memory 28, telemetry module 30, fluid delivery pump 32, reservoir 34, refill port 36, internal passages 38, and power source 44. Processor 26 is communicatively connected to memory 28, telemetry module 30, and fluid delivery pump 32. Fluid delivery pump 32 is connected to reservoir 34 and internal passages 38. As will be described in greater detail below, reservoir 34 used in conjunction with MIP 12 may be an external reservoir of varying types removably connected to MIP 12 or may be a chamber within MIP 12 that is configured to store a therapeutic agent. MIP 12 also includes power source 44, which is configured to deliver operating power to various components of the MIP.

In some examples, MIP 12 may include a plurality of reservoirs for storing more than one type of therapeutic agent. However, for ease of description, a MIP 12 including a single reservoir 34 is primarily described with reference to the disclosed examples.

During operation of MIP 12, processor 26 controls fluid delivery pump 32 with the aid of instructions associated with program information that is stored in memory 28 to deliver a therapeutic agent from reservoir 34 to test subject 16 via catheter 18. As will be described in detail below, fluid delivery pump 32 includes an electromagnetic piston pump configured to cycle through a large number of high frequency pump strokes to deliver an accurate, metered volume of fluid to test subjects. Instructions executed by processor 26 may, for example, define therapy programs that specify the dose of therapeutic agent that is delivered to a target tissue site within test subject 16 from reservoir 34 via catheter 18. The programs may further specify a schedule of different therapeutic agent rates and/or other parameters by which MIP 12 delivers therapy to test subject 16. Therapy programs may be a part of a program group, where the group includes a number of therapy programs. Memory 28 of MIP 12 may store one or more therapy programs and/or program groups, as well as other parameters related to the operation of MIP 12 or the testing of subject 16. A clinician may select and/or generate additional therapy programs for use by MIP 12, e.g., via external programmer 20 at any time during therapy or as designated by the clinician.

Because MIP 12 includes programmable electronics, e.g. processor 26 and memory 28 and telemetry module 30, the device may be programmed according to different parameters multiple times during a single animal test or across multiple tests. The flexible and robust programmability of MIP 12 provides a number of advantages over prior pumps employed in pre-clinical animal studies, which commonly are completely passive (e.g. osmotic) or can only be programmed once prior to implantation. The programmability of MIP 12 may allow for more sophisticated study designs such as designs where an infusion is triggered after some sort of behavior out of the animal. For example, if a test subject fitted with MIP 12 performs a certain task, a telemetry command could be triggered which delivers a bolus as a kind of reward to the subject.

In one example, MIP 12 may function as a “slave” device only operating to deliver therapeutic agent to test subject 16 or execute other functions under instruction from an external device, e.g. programmer 20 via instructions transmitted by telemetry module 30.

Components described as processors within MIP 12, external programmer 20, or any other device described in this disclosure may each include one or more processors, such as one or more microprocessors, digital signal processors (DSPs), application specific integrated circuits (ASICs), field programmable gate arrays (FPGAs), programmable logic circuitry, or the like, either alone or in any suitable combination.

In some examples, processor 26 may not directly control fluid delivery pump 32. For example, MIP 12 may include pump control circuitry that is configured to control pump 32. In one example, pump control circuitry included in MIP 12 may include a switched-capacitor charge pump to indirectly power the high-current electromagnetic pump from a low-current power source 44, e.g. a low-current battery.

Memory 28 of MIP 12 may store instructions for execution by processor 26 including, e.g., therapy programs and/or program groups and any other information regarding therapy delivered to test subject 16 and/or the operation of MIP 12. Memory 28 may include separate memories for storing instructions, test subject information, therapy parameters, therapy adjustment information, program histories, and other categories of information such as any other data that may benefit from separate physical memory modules. Therapy adjustment information may include information relating to timing, frequency, and rate adjustments.

At various times during the operation of MIP 12 to treat test subject 16, communication to and from MIP 12 may be necessary to, e.g., change therapy programs, adjust parameters within one or more programs, or to otherwise download information to or from MIP 12. Processor 26 may control telemetry module 30 to wirelessly communicate between MIP 12 and one or more other devices including, e.g. programmer 20. Telemetry module 30 in MIP 12, as well as telemetry modules in other devices described in this disclosure, such as programmer 20, can be configured to use RF communication techniques to wirelessly send and receive information to and from other devices. In addition, telemetry module 30 may communicate with programmer 20 via proximal inductive interaction between MIP 12 and the external programmer. Telemetry module 30 may send information to external programmer 20 on a continuous basis, at periodic intervals, or upon request from the programmer.

Power source 44 delivers operating power to various components of MIP 12. Power source 44 may include a small rechargeable or non-rechargeable battery and a power generation circuit to produce the operating power. In the case of a rechargeable battery, recharging may be accomplished through proximal inductive interaction between an external charger and an inductive charging coil within MIP 12. In some examples, power requirements may be small enough to allow MIP 12 to utilize test subject motion and implement a kinetic energy-scavenging device to trickle charge a rechargeable battery. In other examples, traditional batteries may be used for a limited period of time. As another alternative, an external inductive power supply could transcutaneously power MIP 12 as needed or desired.

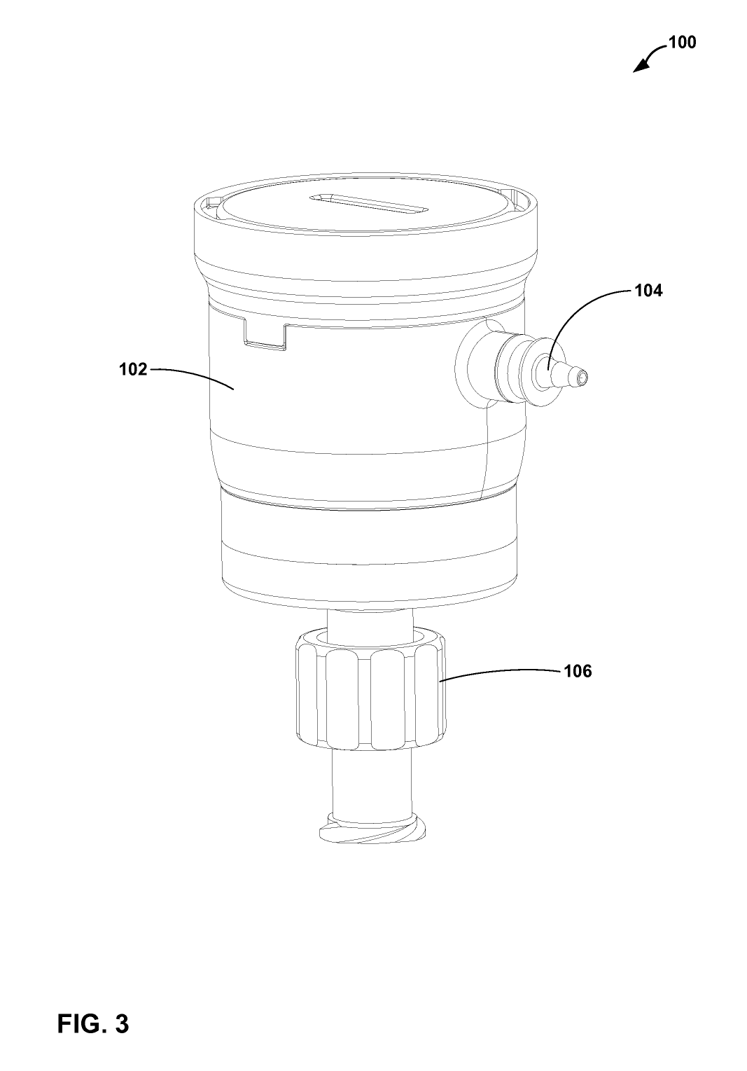

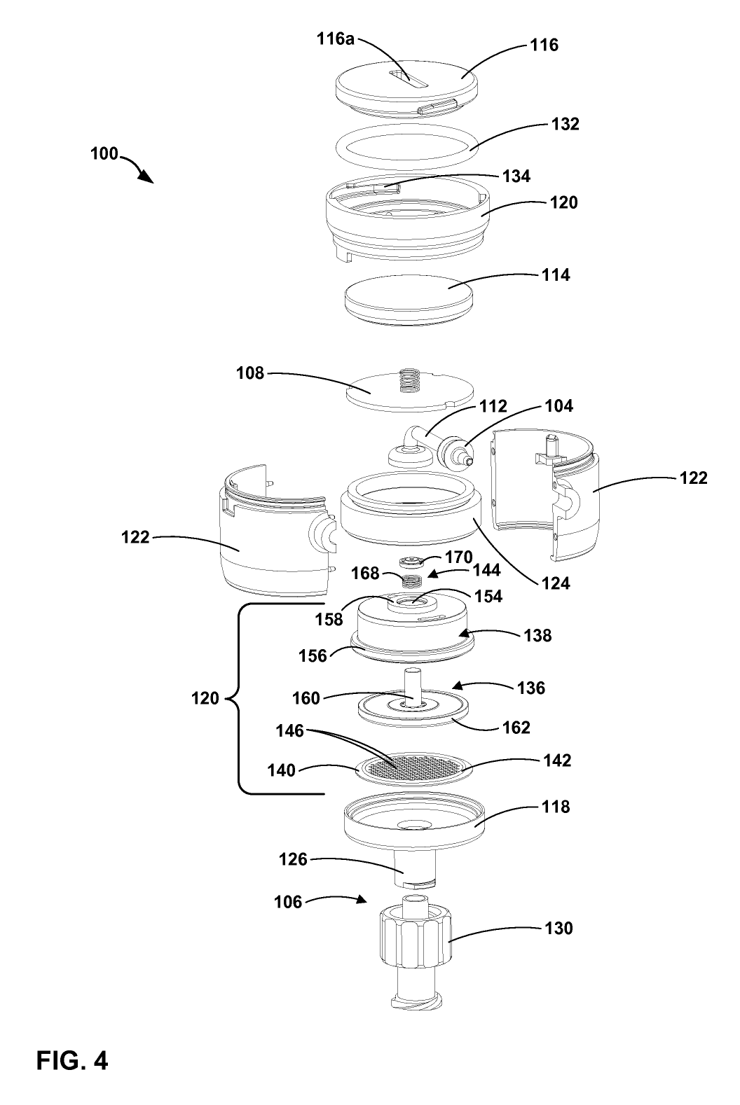

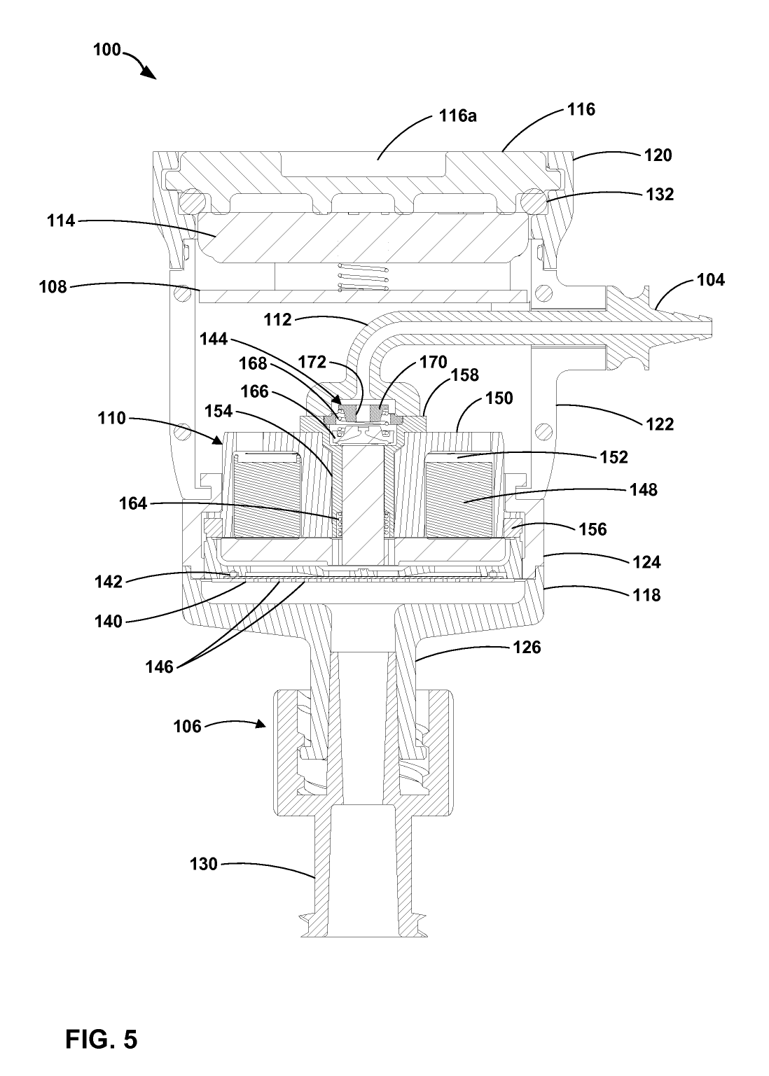

FIGS. 3-5 illustrate an example configuration of a MIP in accordance with this disclosure. FIG. 3 is a perspective view of example MIP 100. FIG. 4 is an exploded view of MIP 100. And, FIG. 5 is a section view of MIP 100.

Referring to FIG. 3, MIP 100 includes housing 102, catheter junction 104, and reservoir junction 106. Housing 102 may contain the pump and control electronics, as well as various other components of MIP 100. Housing 102 may be constructed from biocompatible materials that resist corrosion and degradation from bodily fluids including, e.g., titanium or biologically inert polymers. Housing may be fabricated using a variety of solid material manufacturing techniques, including, e.g. pressing, casting, molding, or any one or more of various material removal processes, including, e.g., milling, turning, grinding, electrical discharge machining (EDM), or laser or torch cutting. In an example in which part or all of housing 102 is fabricated from a plastic, part or all of housing 102 may be manufactured using injection molding techniques.

Catheter junction 104 protrudes from one side of housing 102 and is configured to couple MIP 100 to various types of catheters, including, e.g. a partially or completely implantable catheter, e.g., percutaneous catheter 18 illustrated in FIG. 1. In other examples, catheter junction 104 may be arranged differently with respect to housing 102 of MIP 100, including, e.g. protruding from the end of MIP 100 generally opposite reservoir junction 106.

Reservoir junction 106 protrudes from one end of MIP 100 and is configured to fluidically connect the pump of MIP 100 to a reservoir configured to store a therapeutic agent for delivery to a non-human test subject. Reservoir junction 106 may include a universal connector configured to fluidically connect a plurality of different types of reservoirs to MIP 100. In the example of FIGS. 3-5, reservoir junction 106 includes a Luer connector. A Luer connector is a fluidic connection device including a male and female tapered junction designed to form a sealed fluidic connection between two components. Leur connectors are universal connectors that allow for connection to a multitude of different types of reservoirs options and which therefore may provide a great deal of flexibility in pre-clinical study design. There are multiple types of Luer connectors including lock and slip connectors, the lock type generally including a threaded connection and the slip generally including a press-fit connection. As with the catheter junction 104, in other examples, reservoir junction 106 may be arranged differently with respect to housing 102 of MIP 100, including, e.g. protruding from the side or the other end of MIP 100.

The configuration and components of MIP 100 are shown in greater detail in the exploded view of FIG. 4 and section view of FIG. 5. Referring to FIGS. 4 and 5, MIP 100 also includes circuit board 108, pump 110, conduit 112, and battery 114, all of which are sealed within housing 102. Circuit board 108 and battery 114 are arranged toward one end of housing 102 generally opposite the other end toward which pump 110 is arranged. Conduit 112, which is connected to catheter junction 104, is interposed between circuit board 108 and battery 114 and pump 110. Pump 110 is an electromagnetic piston pump including an inlet fluidically connected to reservoir junction 106 and an outlet fluidically connected to conduit 112, which is connected to catheter junction 104.

The configuration and arrangement of housing 102, catheter junction 104, and reservoir junction 106 are also illustrated in greater detail in FIGS. 4 and 5. Housing 102, for example, includes a number of portions, including first end 116, second end 118, and first, second, and third sections, 120, 122, and 124, respectively, arranged between the first and second ends. Circuit board 108 and battery 114 are arranged within first section 120 toward first end 116 of housing 102 generally opposite second end 118 toward which pump 110 is arranged. Conduit 112 is arranged within second section 122 and pump 110 is arranged within third section 124 of housing 102. It should be noted that the portion of second section 122 within which conduit 112 is arranged includes a relatively large amount of empty space. In another example according to this disclosure, the empty space within second section 122 of housing 102 may be utilized for addition functions other than a place holder for conduit 112. For example, an internal reservoir for a therapeutic agent may be arranged in this space within second section 112 of housing 102. In any event, second end 118 of housing 102 forms the inlet to pump 110 and part of reservoir junction 106, including female portion 126 of the universal Luer reservoir junction 106 to which the male portion 130 of the connector joins to form a sealed fluidic connection that may be connected to an external reservoir.

Depending on the application, e.g. depending on whether MIP 100 is configured to be harnessed to or implanted within a test subject, the various portions of housing 102 of MIP 100 may be joined in different ways to form different seals between an external environment and the internal components of the MIP, e.g. between bodily fluids and circuit board 108, pump 110, conduit 112, and battery 114. For example, first end 116, second end 118, and first, second, and third sections, 120, 122, and 124, respectively, of housing 102 may be joined with one or more of O-rings or other removable seals, medical adhesives, or welds. As illustrated in FIGS. 4 and 5, first end 116 of housing 102 is joined to first section 120 via a thread in first section 120 and O-ring 132. Second and third sections, 122 and 124, respectively, and second end 118 of housing 102 may be connected via one or more medical adhesives and/or by welding the sections together. Regardless of the particular combination of techniques and components, the manner in which housing 102 is assembled to seal circuit board 108, pump 110, conduit 112, and battery 114 therein may be configured, in some examples, to provide a hermitic seal between the external environment and the internal components of MIP 100.

To increase longevity and improve reusability of MIP 100, circuit board 108 and battery 114 are configured to be easily removed from the device for repair or replacement. For example, circuit board 108 and battery 114 may be stacked within first section 120 of housing 102 and sealed therein by removable first end 116 and O-ring 132. First end 116 may be configured to engage threads 134 in first section 120 of housing 102 to be tightened into and loosened from engagement with O-ring 132. In the event, one or more of circuit board 108 and battery 114 become damaged, or if battery 114 loses charge, first end 116 may be removed from housing 102 by unthreading the end from first section 120. First end 116 may include slot 116a configured for engagement by a tool or by, e.g., a clinician's or other operator's fingernail to unthread first end 116 from first section 120 of housing 102. In other examples, first end 116 may be configured differently for removal from housing 102 including, e.g., being configured for engagement by different tools, e.g. Phillips, Fearson, hexagonal, and hexalobular socket (also known as Torx) drivers.

Circuit board 108 may include various programmable electronics that are configured to control pump 110 to deliver therapeutic agents to test subjects. For example, circuit board 108 may include one or more processors, memory, and telemetry components. In one example, circuit board 108 includes components similar in structure and function to processor 26, memory 28, and telemetry module 30 described above with reference to FIG. 2.

Battery 114 may generally be configured to power at least circuit board 108 and electromagnetic piston pump 110. Battery 114 may be a rechargeable or primary cell battery or several such batteries. In one example, battery 114 includes a Cfx, CSVO, Zinc Air, Silver Oxide, Lithium Manganese Dioxide, or Lithium Ion battery. In one example, battery 114 comprises a voltage rating of 3 volts. In one example, battery 114 includes a CR2032 coin cell battery rated for 3 volts and 240 milliamp-hours capacity at approximately 200 microamps. In such an example, battery 114 may be capable of powering pump 110 of MIP 100 to deliver approximately 40 milliliters of a therapeutic agent or power operation of the pump at about 100 microliters per day for 1 year.

In one example, battery 114 may not directly power certain components of MIP 100. For example, battery 114 may not directly power pump 110, but, instead, battery 114 may power capacitor at low current which is then used to power the electromagnetic pump in a short, high-current pulse.

As noted above, reservoir junction 106 includes a universal Luer connector, which is a fluidic connection device including male and female tapered portions 130 and 126, respectively, designed to form a sealed fluidic connection between a various types of removable reservoirs and the inlet to pump 110 of MIP 100. Universal Luer reservoir junction 106 includes a lock type connector, which includes a threaded connection between male and female portions 130 and 126, respectively. Various reservoirs may be employed in conjunction with MIP 100 and other MIPs according to this disclosure. In one example, a flexible, refillable bag may be fluidically connected to MIP 100 via Luer reservoir junction 106. In another example, a rigid chamber may be connected to or formed as part of MIP 100. In the case of a rigid reservoir chamber connected to or incorporated in MIP 100, the reservoir may include a refill port, including, e.g. a self-sealing membrane, or septum to prevent loss of therapeutic agent delivered to the reservoir via the refill port. For example, after a hypodermic needle penetrates the membrane of the refill port and the reservoir is filled with a therapeutic agent or other substance (e.g. saline), the membrane may seal shut when the needle is removed from the refill port. In another example, a glass syringe or tubing connected to a bellows or other reservoir may be fluidically connected to MIP 100 via Luer reservoir junction 106.

MIP 100 also includes electromagnetic piston pump 110, which is configured to deliver a therapeutic agent from a reservoir to a target delivery site within a test subject. Piston pump 110 includes piston/pole assembly 136, coil assembly 138, cover 140, O-ring 142, and check valve 144. The inlet of piston pump 110 is defined by cover 140, which includes holes 146 and is configured to be received in second end 118 of housing 102. The outlet of pump 110 includes check valve 144. During the operation of pump 110, therapeutic agent flows through holes 146 in cover 140 into an enclosure of the pump. Once within the enclosure under cover 140, the agent is pushed by piston/pole assembly 136 through check valve 144. After passing through valve 144, the therapeutic agent is directed to one or more target sites within a test subject, e.g. via conduit 112 and a catheter connected to catheter junction 104. In some examples, filter element 141 is interposed between piston/pole assembly 136 and cover 140 and, when assembled in pump 110, O-ring gasket 142 forms a seal between the filter element and cover to prevent any therapeutic agent flowing through pump 110 from bypassing the filter element.

Coil assembly 138 of electromagnetic piston pump 110 includes electromagnetic coil 148 and magnetic cup 150. Magnetic cup 150 forms a recess 152 and central aperture 154. Recess 152 of magnetic cup 150 is sized and shaped to receive electromagnetic coil 148. Central aperture 154 defines part of the flow path through which piston/pole assembly 136 pumps therapeutic agent through check valve 144. Magnetic cup 150 may be fabricated from a highly magnetic material. The highly magnetic material of magnetic cup 150 efficiently magnetizes in response to current through electromagnetic coil 148. As an example, magnetic cup 150 may include a highly magnetic steel alloy. As another example, magnetic cup 150 may include a highly magnetic stainless steel alloy such as 430F or 430FR. However, as highly magnetic materials are generally susceptible to corrosion, in some examples, magnetic cup 150 may be separated from the flow path of fluid being pumped by pump 110 to prevent corrosion of magnetic cup 150. For example, magnetic cup 150 and electromagnetic coil 148 may be separated from the flow path of pump 110 at least in part by a barrier plate coupled to coil assembly 138, e.g. welded to the assembly. In some examples, magnetic cup 150 may include weld ring 156 and sleeve 158, which are joined to magnetic cup and provide a material structure to which a barrier plate may be hermetically sealed.

Electromagnetic coil 148 includes one or more insulated conductors arranged in a multitude of turns. As examples, electromagnetic coil 148 may include a single continuous conductor or more than one conductor electrically connected in series or in parallel. Electromagnetic coil 148 may be connected to a flex circuit that provides the electrical connections used to deliver current to electromagnetic coil 148 from battery 114. Within fluid delivery pump 110, delivering current to electromagnetic coil 148 magnetizes magnetic cup 150 in order to attract pole 162 of piston/pole assembly 136, which, in turn, drives piston 160 to generate a pump stroke of pump 110.

Piston/pole assembly 136 includes piston 160 and pole 162. Piston/pole assembly 136 is positioned such that piston 160 is located within sleeve 158 arranged in central aperture 154 of magnetic cup 150. Piston pump 110 also includes piston spring 164, which is located within sleeve 158 adjacent one end of piston 160. Piston spring 164 functions to bias pole 162 away from electromagnetic coil 148 and magnetic cup 150. Piston 160 may be interference fit to pole 162 or secured to pole 162 by other suitable techniques. Pole 162 comprises a magnetic material that is attracted to magnetic cup 150 to produce a pump stroke. Because pole 162 is within the fluid flow path, the material of pole 162 may be configured to resist corrosion. As an example, pole 162 may include a magnetic stainless steel alloy, such as AL29-4. Likewise, piston 160 is also located within the fluid flow path and may therefore also be configured to resist corrosion. As an example, piston 160 may include a sapphire material, which can limit wear between piston and sleeve 158 caused by the pumping action of fluid delivery pump 110. As other examples, piston 160 may include a metal material, such as a stainless steel or titanium alloy. In some examples, piston/pole assembly 136 may include a unitary component consisting of a single magnetic material such as a stainless steel alloy.

Piston/pole assembly 136 actuates within an enclosure within third section 124 of housing 102 between cover 120 and coil assembly 138. Piston spring 164 biases piston/pole assembly 136 away from check valve 144. The motion of piston/pole assembly 136 is driven by electromagnetic coil 148. Specifically, during a pump stroke, current through electromagnetic coil 148 serves to magnetize magnetic cup 150 to attract pole 162 of piston/pole assembly 136. The magnetic attraction force between pole 162 and magnetic cup 150 overcomes the force of piston spring 164 to create a pumping action of piston 160. The motion of piston 160 forces therapeutic agent w sleeve 158 arranged in central aperture 154 of magnetic cup 150 through check valve 144. Following a pump stroke, current through electromagnetic coil 148 stops, and piston spring 164 returns piston/pole assembly 136 to its original position away from check valve 144.

Therapeutic agent pushed by piston 160 during a pump stroke exits piston pump 110 through check valve 144. Check valve 144 is generally a one-way valve that is configured to allow a therapeutic agent to flow from pump 130 through an exit port of the valve and to substantially prevent flow back into the pump through the exit port. Check valve 144 includes disc 166, valve spring 168, and bonnet 170. Valve spring 168 functions to bias disc 166 against a seat in magnetic cup 150, e.g. in sleeve 158 arranged in aperture 154 of magnetic cup 150. Bonnet 170 functions to hold spring 168 in place. Bonnet 170 includes exit port 172 that provides a fluid passageway through bonnet 170. When check valve 144 is closed, disc 166 seals to the seat in, e.g. in sleeve 158 arranged in aperture 154 of magnetic cup 150. The configuration of check valve 144 may be referred to as a lift check valve. In other examples, different valve configurations may be used including, but not limited to, ball check valves, diaphragm valves, gate valves and other valves. The design of pump 110 allows different valves to be selected depending on, e.g. a particular therapeutic agent to be pumped through and the desired pumping characteristics the pump.

MIP 100 is a device that is suitable for use with a human patient, e.g., suitable for human implantation but is configured for non-human animal testing. MIP 100 is sized to be at least one of harnessed to or implanted in a test subject without substantially altering behavior of the subject. In particular, MIP 100 is sized to be at least one of harnessed to or implanted in a test subject including a weight greater than or equal to approximately 150 to approximately 250 grams, which may include mammals ranging from medium-sized rodents up to, e.g. primates. In one example, MIP 100 includes a weight approximately equal to 30 grams. In one example, MIP 100 includes a volume of approximately 12.6 cubic centimeters (0.77 cubic inches). MIP 100 may include a length in a range from approximately 3.1 to approximately 5.1 centimeters and a width in a range from approximately 1.95 to approximately 2.4 centimeters. In one example, MIP 100 may include a length approximately equal to 3.8 centimeters and a width approximately equal to 2.4 centimeters.

Configuring MIP 100 to be suitable for human implantation or other uses with a human patient may include that the materials, fluid delivery capacity, and/or longevity of MIP 100 are suitable for use in conjunction with an IID implanted within a human patient. For example, electromagnetic piston pump 110 and other components of MIP 100 that interact with the therapeutic agent, e.g. part or all of housing 102 and conduit 112 may be fabricated from materials suitable for use in humans, including titanium or a biologically inert polymer. Additionally, pump 110 may be capable of delivering pump strokes at high frequencies over long periods of time. For example, electromagnetic piston pump 110 MIP 100 may be designed to deliver 1 microliter pump strokes at varying programmable rates over periods of time ranging from a few months (range of time appropriate for animal testing) to five or more years (range of time appropriate for human implantation). Electromagnetic pump 110 of MIP 100 may be configured to deliver as many as 2.5 million pump stroke strokes, totaling approximately 2.5 liters of therapeutic agent, or 2500 milliliters of the agent. A single animal test employing a MIP may include delivery of on the order of approximately 2-3 milliliters of therapeutic agent over the course of the study. Thus, MIP 100 may be configured to deliver a cumulative 2500 milliliters of agent which may be used in more than 850 animal tests. At least one of pump 110, the housing 102, and conduit 112 of MIP 100 may be configured to be resterilized for a plurality of uses with a plurality of non-human test subjects. Thus, MIP 100 may be harnessed to or implanted in a number of test subjects and resterilized in between in order to be employed across a large number of animal tests.

There are a number of characteristics of MIP 100 that may enable the device to be resterilized for multiple uses, e.g. in multiple studies or with multiple subjects in one study. MIP 100 is generally modular in design such that those components that are not capable of being sterilized, e.g. battery 114 and circuit board 108 can be removed and reinstalled or replaced after resterilization. Additionally, the limited use of less robust materials and increased use of more robust materials, e.g. decreased use of polymers and increased use of metals like titanium may make MIP 100 more amendable to sterilization since polymers in the flow path of therapeutic agent through the can absorb material and contaminate future studies. The materials in MIP 100 may also be able to withstand the temperatures encountered in sterilization procedures, e.g. temperatures as high as 125 degrees Celsius.

MIP 100 may also be configured to deliver a wide range of rates and doses of therapeutic agents, thus making the device suitable for both human and non-human testing and/or use. For example, MIP 100 may be configured to deliver small doses of a therapeutic agent at lower rates for animal testing, but may also be driven at much higher frequencies to deliver larger doses at higher rates for use with human patients. In this manner, MIP 100 may be employed for pre-clinical animal testing and be suitable for human implantation.

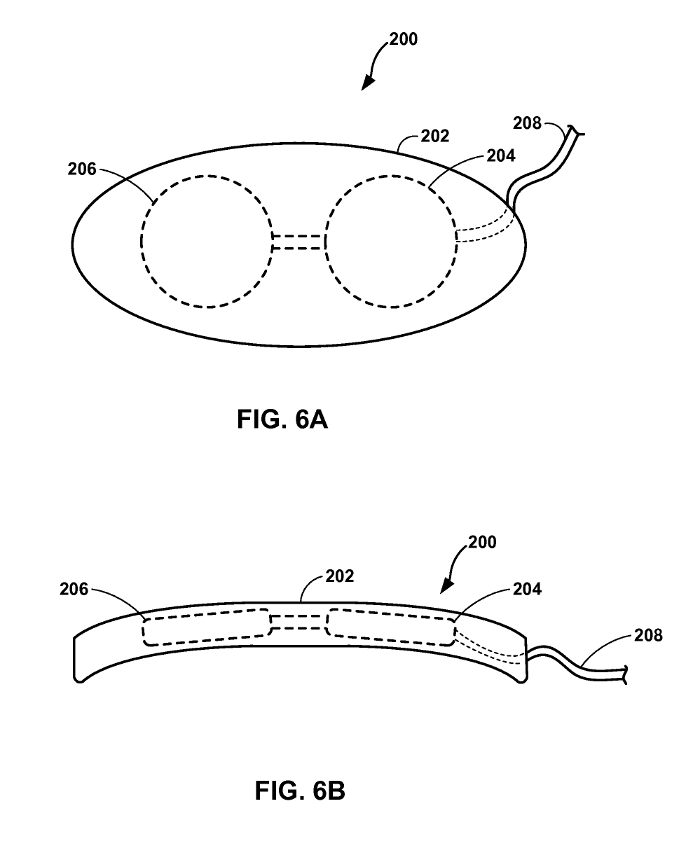

Although the foregoing examples have been described with reference to a MIP including a generally cylindrical shape with a piston pump and circuit board arranged in stacked relationship to one another, in other examples according to this disclosure a MIP suitable for use with a human patient configured for non-human animal testing may include a number of different geometric configurations. For example, FIGS. 6A and 6B are plan and elevation views, respectively, of example MIP 200 according to this disclosure. MIP 200, in contrast to MIP 100, includes a contoured oval shape housing 202 in which electromagnetic piston pump 204 and control electronics 206 are arranged. Control electronics may include, e.g., a circuit board configured to control piston pump 204 to deliver a therapeutic agent through catheter 208, as well as a battery configured to power the circuit board and pump. Pump 204 and electronics 206 are arranged in side-by-side relationship to one another within housing 202 of MIP 200 such that MIP 200 assumes a generally flatter, wider configuration than cylindrical MIP 100. MIP 200 may be contoured and shaped as illustrated in FIGS. 6A and 6B to be implanted within a test subject, e.g. below the skin and surface tissue layers of the test subject.

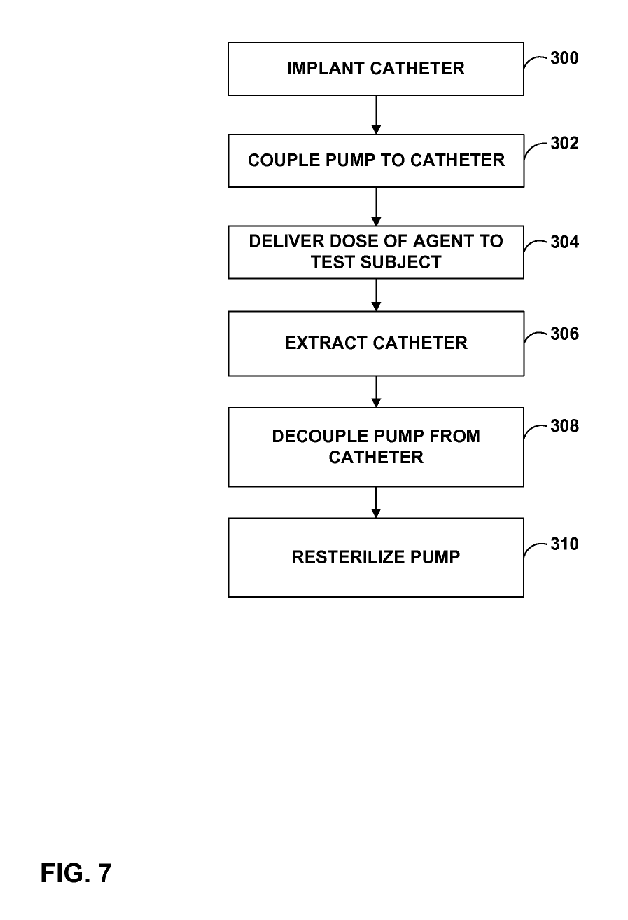

FIG. 7 is a flow chart illustrating an example method of using a MIP suitable for use with a human patient for non-human animal testing. The example method of FIG. 7 includes implanting at least a portion of a catheter within a non-human test subject (300), coupling the MIP to the catheter (302), delivering a dose of the therapeutic agent to the test subject with the MIP (304), and, optionally, extracting the catheter from the non-human test subject (306), decoupling the MIP from the catheter (308), and resterilizing the MIP (310). The test subject in association with which the method of FIG. 7 may be employed includes a weight greater than or equal to approximately 150 to approximately 250 grams.

FIG. 7 may be employed with any MIP in accordance with this disclosure. As such, a MIP employed in the example method of FIG. 7 includes an electromagnetic piston pump, a circuit board, and a housing within which the pump and the circuit board are sealed. The electromagnetic piston pump is configured to deliver a therapeutic agent from a reservoir through an implantable catheter. The circuit board includes programmable electronics configured to control the pump to deliver the therapeutic agent through the catheter. The MIP is sized to be at least one of harnessed to or implanted in a test subject including a weight greater than or equal to approximately 150 to approximately 250 grams. For example, the method of FIG. 7 may be employed using any one of MIP 12, MIP 100, or MIP 200 described above.

The method of FIG. 7 includes the steps of implanting at least a portion of a catheter within a non-human test subject (300), coupling the MIP to the catheter (302), delivering a dose of the therapeutic agent to the test subject with the MIP (304). For example, percutaneous catheter 18 may be implanted within test subject 16 via incision 17 and MIP 12 may coupled to the catheter and harnessed to the test subject as illustrated in FIG. 1. Processor 26 may control electromagnetic piston pump 32 of MIP 12, e.g. based on instructions stored in memory 28, to deliver therapeutic agent from reservoir 34 through catheter 18 to a target site within test subject 16.

Pump 32 may be configured to deliver the agent to test subject 16 in 1 micro liter pump strokes at high frequencies, if necessary. In the course of testing the therapeutic agent and MIP 12, pump 32 may deliver on the order of 2-3 milliliters before the test is complete. After completing the testing on test subject 16, catheter 18 may be extracted from the test subject (306) and MIP 12 may be decoupled from the catheter (308), before or after extraction. Additionally, as indicated in FIG. 7, one or more components of MIP 12 may be resterilized, e.g. for use on another test subject. For example, one or more of the housing, electromagnetic piston pump, and fluid flow conduit of MIP 12 may be resterilized after completing testing on test subject 16.

Because of the longevity, reusability, and delivery capacity of MIP 12, the device may be used across a large number of animal tests before being retired from service. As such, in some examples, the steps of implanting a catheter (300), coupling the MIP to the catheter (302), delivering a dose of the therapeutic agent with the MIP (304), extracting the catheter (306), decoupling the MIP from the catheter (308), and resterilizing the MIP (310) may be repeated until the MIP has cumulatively delivered in a range from approximately 10 milliliters to approximately 2.5 liters of the therapeutic agent, which may include as many as or more than 850 different animal tests.

Techniques described in this disclosure associated with control electronics of a MIP or external device, such as an external programmer may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A miniature infusion pump (MIP) suitable for use with a human patient configured for non-human animal testing, the MIP comprising:
a cylindrical housing;
an electromagnetic piston pump configured to deliver a therapeutic agent from a reservoir through an implantable catheter;
a circuit board comprising programmable electronics configured to control the pump to deliver the therapeutic agent through the catheter, wherein the pump and the circuit board are arranged in stacked relationship to one another within the cylindrical housing such that the pump is arranged toward one end of the cylindrical housing and the circuit board is arranged toward an opposite end of the cylindrical housing;
a conduit interposed between the pump and the circuit board, wherein the conduit is configured to fluidically connect an outlet of the pump to the implantable catheter; and
a reservoir junction connected to the end of the housing toward which the pump is arranged, wherein the reservoir junction is configured to fluidically connect an inlet of the pump to a reservoir configured to store the therapeutic agent.

2. The MIP of claim 1 comprising a weight approximately equal to 30 grams.

3. The MIP of claim 1 comprising a length in a range from approximately 3.1 to approximately 5.1 centimeters and a width in a range from approximately 1.95 to approximately 2.4 centimeters.

4. The MIP of claim 1 comprising a volume approximately equal to 12.6 cubic centimeters (0.77 cubic inches).

5. The MIP of claim 1 further comprising a catheter junction protruding from the housing and fluidically connected to the conduit, wherein the catheter junction is configured to couple the implantable catheter to the MIP.

6. The MIP of claim 1, wherein the conduit comprises a material suitable for human implantation, comprising at least one of titanium or a biologically inert polymer.

7. The MIP of claim 1, wherein the reservoir junction comprises a universal connector configured to fluidically connect a plurality of different types of reservoirs to the inlet of the pump.

8. The MIP of claim 7, wherein the universal connector comprises a luer connector.

9. The MIP of claim 1, wherein at least one of the pump, the housing, and the conduit are configured to be resterilized for a plurality of uses with a plurality of non-human test subjects.

10. The MIP of claim 1, wherein the pump and the circuit board are hermetically sealed within the housing.

11. The MIP of claim 1, further comprising battery arranged within the housing and configured to power at least the circuit board and the pump.

12. The MIP of claim 11, wherein the circuit board and the battery are arranged in stacked relationship within the cylindrical housing such that the battery is interposed between the circuit board and the end of the cylindrical housing toward which the circuit board is arranged.

13. The MIP of claim 11, wherein at least one of the battery and the circuit board are configured to be removable from the housing.

14. The MIP of claim 1, wherein the circuit board comprises a telemetry module configured to transmit information between the programmable electronics and a remote electronic device.

15. The MIP of claim 1, wherein the circuit board comprises memory configured to store information for use by the programmable electronics to control the pump to deliver the therapeutic agent through the catheter.

16. The MIP of claim 1, wherein the pump is configured to cycle through approximately 2.5 million piston strokes.

17. The MIP of claim 1, wherein the pump comprises a nominal 1 microliter per stroke capacity of the therapeutic agent and a cumulative delivery capacity of the therapeutic agent over a plurality of piston stroke cycles in a range from approximately 10 milliliters to approximately 2.5 liters.

18. A method of using a miniature infusion pump (MIP) suitable for use with a human patient for non-human animal testing, the method comprising:
implanting at least a portion of a catheter within a non-human test subject, wherein the catheter is coupled to the MIP and the MIP comprises:
an electromagnetic piston pump configured to deliver a therapeutic agent from a reservoir through the catheter;
a circuit board comprising programmable electronics configured to control the pump to deliver the therapeutic agent through the catheter; and
a housing within which the pump and the circuit board are sealed, and delivering a dose of the therapeutic agent to the test subject through the catheter with the MIP,
wherein the test subject comprises a weight greater than or equal to approximately 150 to approximately 250 grams.

19. The method of claim 18, further comprising:
extracting the catheter from the non-human test subject;
decoupling the MIP from the catheter; and
resterilizing the MIP.

20. The method of claim 19, further comprising:
implanting at least a portion of a second catheter within a second non-human test subject, wherein the second catheter is configured for human implantation;
coupling the MIP to the second catheter; and
delivering a dose of the therapeutic agent to the second test subject with the MIP via the catheter.

21. The method of claim 20, further comprising repeating the steps of extracting the catheter, decoupling the MIP, resterilizing the MIP, implanting another catheter, coupling the MIP, and delivering a dose of the therapeutic agent until the MIP has cumulatively delivered in a range from approximately 10 milliliters to approximately 2.5 liters of the therapeutic agent.

22. The method of claim 20, further comprising repeating the steps of extracting the catheter, decoupling the MIP, resterilizing the MIP, implanting another catheter, coupling the MIP, and delivering a dose of the therapeutic agent until the pump of the MIP has cycled through approximately 2.5 million piston strokes.

23. The method of claim 18, wherein the MIP comprises a battery arranged within the housing and configured to power at least the circuit board and the pump and further comprising removing at least one of the battery and the circuit board from the housing.

24. A miniature infusion pump (MIP) suitable for use with a human patient configured for non-human animal testing, the MIP comprising:
an electromagnetic piston pump configured to deliver a therapeutic agent from a reservoir through an implantable catheter;
a circuit board comprising programmable electronics configured to control the pump to deliver the therapeutic agent through the catheter; and
a housing within which the pump and the circuit board are sealed,
wherein the MIP is sized to be at least one of harnessed to or implanted in a non-human test subject comprising a weight greater than or equal to approximately 150 to approximately 250 grams.

25. The MIP of claim 24 comprising a weight approximately equal to 30 grams.

26. The MIP of claim 24 comprising a length in a range from approximately 3.1 to approximately 5.1 centimeters and a width in a range from approximately 1.95 to approximately 2.4 centimeters.

27. The MIP of claim 24 comprising a volume approximately equal to 12.6 cubic centimeters (0.77 cubic inches).

28. The MIP of claim 24, wherein the housing comprises a generally cylindrical housing and the pump and the circuit board are arranged in stacked relationship to one another within the cylindrical housing such that the pump is arranged toward one end of the cylindrical housing and the circuit board is arranged toward an opposite end of the cylindrical housing.

29. The MIP of claim 28, further comprising a conduit interposed between the pump and the circuit board, wherein the conduit is configured to fluidically connect an outlet of the pump to the implantable catheter.

30. The MIP of claim 29, further comprising a reservoir junction connected to the end of the housing toward which the pump is arranged, wherein the reservoir junction is configured to fluidically connect an inlet of the pump to a reservoir configured to store the therapeutic agent.

31. The MIP of claim 24, wherein the pump is configured to cycle through approximately 2.5 million piston strokes.

32. The MIP of claim 24, wherein the pump comprises a nominal 1 microliter per stroke capacity of the therapeutic agent and a cumulative delivery capacity of the therapeutic agent over a plurality of piston stroke cycles in a range from approximately 10 milliliters to approximately 2.5 liters.

33. The MIP of claim 24, wherein the pump and the circuit board are arranged in side-by-side relationship to one another within the housing.
